(19) United States
(12) Patent Application Publication
(10) Pub. No.: US 2025/0001133 A1
(43) Pub. Date: Jan. 2, 2025

(54) STEERABLE CATHETER WITH PUSH RING ASSEMBLY

(71) Applicant: Boston Scientific Scimed, Inc., Maple Grove, MN (US)

(72) Inventors: David Cory Kirt, Minnetonka, MN (US); Kevin Martin, Maple Grove, MN (US)

(73) Assignee: Boston Scientific Scimed, Inc., Maple Grove, MN (US)

(21) Appl. No.: 18/758,380

(22) Filed: Jun. 28, 2024

Related U.S. Application Data
(60) Provisional application No. 63/524,546, filed on Jun. 30, 2023.

(51) Int. Cl. A61M 25/01

(52) U.S. Cl. CPC A61M 25/0113

(57) ABSTRACT

A steerable catheter includes an outer sheath defining an outer sheath lumen extending from a proximal region to a distal region. A push ring is secured within the proximal region of the outer sheath lumen. A pull ring is secured within the distal region of the outer sheath lumen. An inner shaft member extends within the outer sheath lumen between the push ring and the pull ring. A pair of push members extend through the outer sheath lumen and secured to the push ring, each of the pair of push members defining a push member lumen extending therethrough. A pair of pull wires are secured to the pull ring, each of the pair of pull wires extending through one of the push member lumens.

### CROSS-REFERENCE TO RELATED APPLICATIONS

This application claims the benefit of priority under 35 U.S.C. § 119 of U.S. Provisional Application No. 63/524,546, filed Jun. 30, 2023, the entire disclosure of which is hereby incorporated by reference.

### TECHNICAL FIELD

The present disclosure pertains to medical devices, and methods for manufacturing medical devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to several alternative designs, materials and methods of manufacturing medical device structures and assemblies, and the use thereof. An example may be found in a steerable catheter. The steerable catheter includes an outer sheath defining an outer sheath haft lumen extending from a proximal region to a distal region, a push ring secured within the proximal region of the outer sheath lumen, a pull ring secured within the distal region of the outer sheath lumen and an inner shaft member extending within the outer sheath lumen between the push ring and the pull ring. A pair of push members extend through the outer sheath lumen and are secured to the push ring, each of the pair of push members defining a push member lumen extending therethrough. A pair of pull wires are secured to the pull ring, each of the pair of pull wires extending through one of the push member lumens.

Alternatively or additionally, each of the pull wires may extend within the outer sheath lumen proximal of the pair of push members.

Alternatively or additionally, each of the pull wires may extend within the outer sheath lumen distal of the pair of push members.

Alternatively or additionally, the push ring may include a metal cylinder and each of the push members may be welded to the metal cylinder.

Alternatively or additionally, each of the push members may include a tightly wound coil.

Alternatively or additionally, each of the pull wires, extending through an associated push member, together with the associated push member, may be considered a Bowden cable.

Alternatively or additionally, the push ring may define an inner diameter, and each push member of the pair of push members may be disposed inside of the inner diameter.

Alternatively or additionally, the push ring may define an inner surface, and each push member of the pair of push members may be disposed radially inwardly from the inner surface.

Alternatively or additionally, the push ring may include a plurality of apertures for securing the push ring to the outer sheath.

Alternatively or additionally, the inner shaft member may include a plurality of articulation joints.

Alternatively or additionally, a first articulation joint proximate the push ring may include a proximal reduced diameter segment adapted to fit inside of the push ring.

Alternatively or additionally, the push ring may include a distally extending first snap fit feature and a first articulation joint proximate the push ring may include a complementary second snap fit feature adapted to form a snap fit with the distally extending first snap fit feature.

Another example may be found in a steering assembly adapted for use in a steerable catheter including an outer sheath defining an outer sheath lumen extending therethrough, the steering assembly adapted to fit within the outer sheath lumen. The steering assembly includes a push ring and a pair of push members welded to the push ring and adapted to extend proximally therefrom, each of the pair of push members defining a push member lumen extending therethrough. The steering assembly includes a pull ring and a pair of pull wires welded to the pull ring and adapted to extend proximally from the pull ring. A force transmission member extends between the push ring and the pull ring. The pair of pull wires each extend through a corresponding push member lumen proximal of the push ring.

Alternatively or additionally, each of the push members include a tightly wound coil.

Alternatively or additionally, each of the pull wires, extending through an associated push member, together with the associated push member, may be considered a Bowden cable.

Alternatively or additionally, the push ring may include a plurality of apertures for securing the push ring to the outer shaft.

Alternatively or additionally, the force transmission member may include a plurality of articulation joints.

Alternatively or additionally, a first articulation joint proximate the push ring may include a proximal reduced diameter segment adapted to fit inside of the push ring.

Alternatively or additionally, the push ring may include a distally extending first snap fit feature and a first articulation joint proximate the push ring may include a complementary second snap fit feature adapted to form a snap fit with the distally extending first snap fit feature.

Another example may be found in a steerable catheter. The steerable catheter includes an outer shaft defining an outer shaft lumen extending from a proximal region to a distal region, a push ring secured within the proximal region of the outer shaft lumen, a pull ring secured within the distal region of the outer shaft lumen, a plurality of articulation joints extending within the outer shaft lumen between the push ring and the pull ring, a pair of push members extending through the outer shaft lumen and welded to the push ring, each of the pair of push members defining a push member lumen extending therethrough, and a pair of pull wires welded to the pull ring, each of the pair of pull wires extending through one of the push member lumens, each of the pair of pull wires extending through the outer shaft lumen both proximal and distal of the pair of push members.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic view of an illustrative steerable catheter shown in a straight configuration;

FIG. 2 is a schematic view of the illustrative steerable catheter of FIG. 1, shown in a deflected configuration;

FIG. 3 is a cross-sectional view taken along the line 3-3 of FIG. 1;

FIG. 4 is a perspective view of a portion of the illustrative steerable catheter of FIG. 1;

FIG. 5 is an enlarged view of a portion of FIG. 4;

FIG. 6 is a schematic view of an illustrative steerable assembly that forms part of the steerable catheter of FIG. 1;

FIG. 7 is a schematic view of a portion of the illustrative steerable catheter of FIG. 1;

FIG. 8 is a perspective view of a portion of the illustrative steerable catheter of FIG. 1;

FIG. 9 is an end view of the portion of the illustrative steerable catheter shown in FIG. 8;

FIG. 10 is a perspective view of a push member secured to a push ring;

FIG. 11 is an end view of FIG. 10;

FIG. 12 is an enlarged view of a portion of FIG. 10;

FIG. 13 is a schematic partial cutaway view of an illustrative steerable catheter;

FIG. 14 is a schematic view of an illustrative connection between a push ring and an articulating joint; and

FIG. 15 is a schematic view of an illustrative connection between a push ring and an articulating joint.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

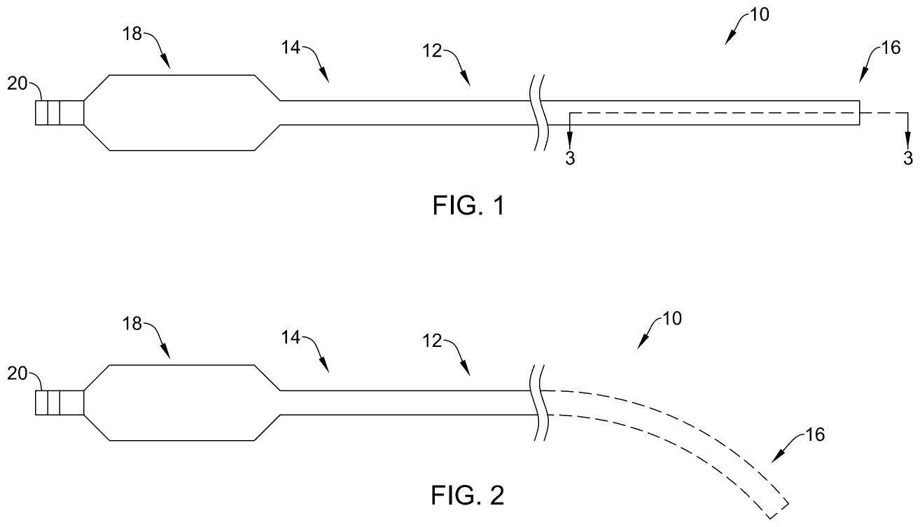

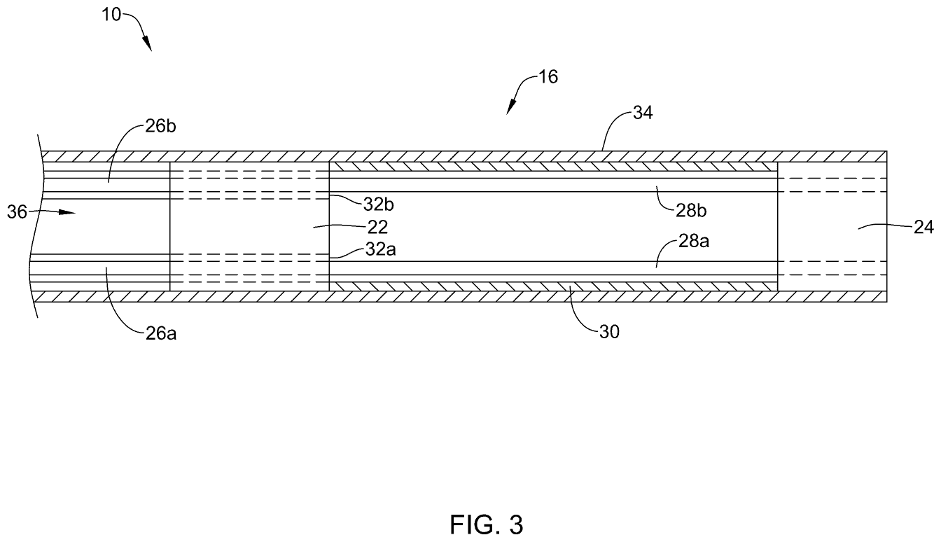

FIGS. 1 and 2 provide schematic views of an illustrative steerable catheter 10. The illustrative steerable catheter 10 may generically represent a steerable catheter usable for any of a variety of purposes. The steerable catheter 10 is shown in FIG. 1 in a straight, or undeflected, configuration. The steerable catheter 10 is shown in FIG. 2 in a curved, or deflected, configuration. In some instances, the steerable catheter 10 may biased to the straight configuration shown in FIG. 1 and may be manipulated into the deflected configuration shown in FIG. 2. In some instances, the steerable catheter 10 may be biased to the curved configuration shown in FIG. 2, and may be manipulated into the straight configuration shown in FIG. 1. The steerable catheter 10 includes an elongate shaft 12 extending from a proximal region 14 to a distal region 16. The elongate shaft 12 may include a single polymeric layer, or may include two or more polymeric layers. In some instances, the elongate shaft 12 may include one or more reinforcing members, for example. As will be discussed, the elongate shaft 12 may include structure that enables the steerable catheter 10 to be steered, or to have the distal region 16 deflected as shown in FIG. 2.

The steerable catheter 10 includes a hub 18. While shown schematically, the hub 18 may include any number of distinct connections for coupling other devices to the steerable catheter 10. As shown, the hub 18 includes a Luer fitting 20 that may be used for flushing the steerable catheter 10 prior to use. The Luer fitting 20 may accommodate a guidewire (not shown) extending through the Luer fitting 20 such that the steerable catheter 10 may be advanced over the guidewire. Other hub configurations are also contemplated.

The steerable catheter 10 includes structure that allows the steerable catheter 10 to be moved between the straight configuration shown in FIG. 1 and the curved configuration shown in FIG. 2. In some instances, the steerable catheter 10 may include a combination of push members and pull wires that enable the steerable catheter 10 to be steered. FIG. 3 is a cross-sectional view of the distal region 16 of the steerable catheter 10, taken along the line 3-3 of FIG. 1. As can be seen in FIG. 3, the steerable catheter 10 includes a push ring 22 and a pull ring 24. The push ring 22 is attached to a pair of push members 26a and 26b. In some instances, the push members 26a and 26b are welded to the push ring 22. A pair of pull wires 28a and 28b are attached to the pull ring 24. In some instances, the pull wires 28a and 28b are welded to the pull ring 24.

In use, a tensile force may be applied to one or both of the pull wires 28a and 28b in order to deflect the distal region 16. The push ring 22 (and push members 26a and 26b) provide a resistive force to an inner shaft member 30 that extends between the push ring 22 and the pull ring 24. In some instances, the inner shaft member 30 may be considered as being a force transmission member. The inner shaft member 30 may be a reinforced polymeric sleeve, for example. In some instances, the inner shaft member 30 may be a laser cut hypotube that has had voids cut out to increase the flexibility of the inner shaft member 30 while allowing the inner shaft member 30 to resist the compressive forces applied to the inner shaft member 30 by virtue of its position between the push ring 22 and the pull ring 24. In some instances, the inner shaft member 30 may be or otherwise include a plurality of articulation joints, for example.

In some instances, the push member 26a may define a push member lumen 32a extending therethrough and the push member 26b may define a push member lumen 32b extending therethrough. In some instances, each of the pull wires 28a and 28b may extend through the push member lumen 32 through the corresponding push members 26a and 26b. For example, the pull wire 28a may extend through the push member lumen 32a within the push member 26a and the pull wire 28b may extend through the push member lumen 32b within the push member 26b. In some instances, the combination of a pull wire such as the pull wire 26a extending through the push member 26a, or the pull wire 26b extending through the push member 64b, may be referred to as a Bowden cable. In some instances, the push member 26a and the push member 26b may themselves be referred to as Bowden cables.

In some instances, each of the push members 26a and 26b may be formed as tightly wound coils. In some instances, the coil may have an inner diameter in a range of 0.005 inches to 0.025 inches and an outer diameter in a range of 0.007 inches to 0.045 inches, and may be formed from a filar having a diameter in a range of 0.001 inches to 0.01 inches. While not expressly shown, each of the push members 26a and 26b may be embedded in a polymeric layer extending proximally of the push ring 22 as a way of anchoring the push members 26a and 26b in position within the steerable catheter 10. In some instances, the pull wires 28a and 28b may be formed of any suitable material and may have a diameter in a range of 0.004 inches to 0.024 inches, for example.

The steerable catheter 10 includes an outer sheath 34 that extends over the push ring 22, the pull ring 24 and the intervening inner shaft member 30. The outer sheath 34 defines an outer sheath lumen 36 that extends through the outer sheath 34 and that accommodates the push ring 22, the pull ring 24 and the intervening inner shaft member 30 within the outer sheath lumen 36. The outer sheath 34 may be formed of any suitable materials. Illustrative materials for the outer sheath 34 include polymers such as urethanes, PEBAs (polyether block amides) and polyamides. The outer sheath 34 may have any desired thickness such as a thickness in a range of 0.002 inches to 0.025 inches. In some instances, the material or materials used for forming the outer sheath 34 may vary along the length of the steerable catheter 10.

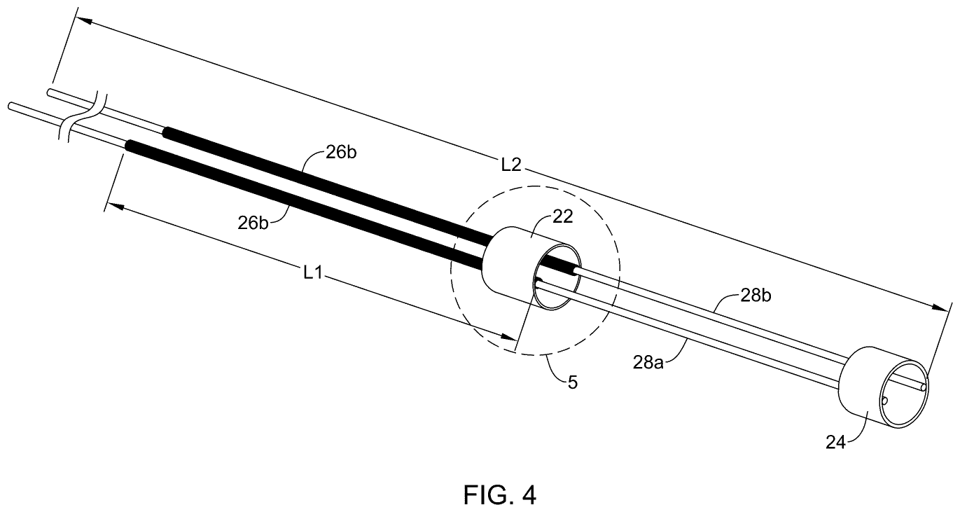

FIG. 4 is a perspective view showing the push ring 22, the pull ring 24, the push members 26a and 26b secured to the push ring 22 and the pull wires 28a and 28b secured to the pull ring 24. As can be seen, the push members 26a and 26b extend proximally from the push ring 22 a length L₁. In some instances, the length L₁ is selected to be long enough to be able to sufficiently anchor the push members 26a and 26b within the outer sheath 34, or within an additional polymeric layer (not shown). In some instances, each of the push members 26a and 26b may have a length L₁ that is in a range of 12 inches to 72 inches. In some instances, the pull wires 28a and 28b have a length L₂ that is sufficient for the pull wires 28a and 28b to extend from the pull ring 24, which is located within the distal region 16 of the elongate shaft 12, to a hub or handle (not shown) that is located at or near a proximal end of the elongate shaft 12 so that the pull wires 28a and 28b may be separately pulled or in combination pulled in order to cause the distal region 16 of the elongate shaft 12 to curve or deflect. In some instances, the pull wires 28a and 28b may have a length L₂ that is in a range of 14 inches to 80 inches, for example.

In some instances, each of the pull wires 28a and 28b may extend within the outer sheath lumen 36 proximal of the push members 26a and 26b. Each of the pull wires 28a and 28b may extend within the outer sheath lumen 36 distal of the push members 26a and 26b. The pull wires 28a and 28b extend through the push member lumens 32a and 32b, respectively. The pull wire 28a is free to translate within the push member lumen 32a extending through the push member 26a. The pull wire 28b is free to translate within the push member lumen 32b extending through the push member 26b.

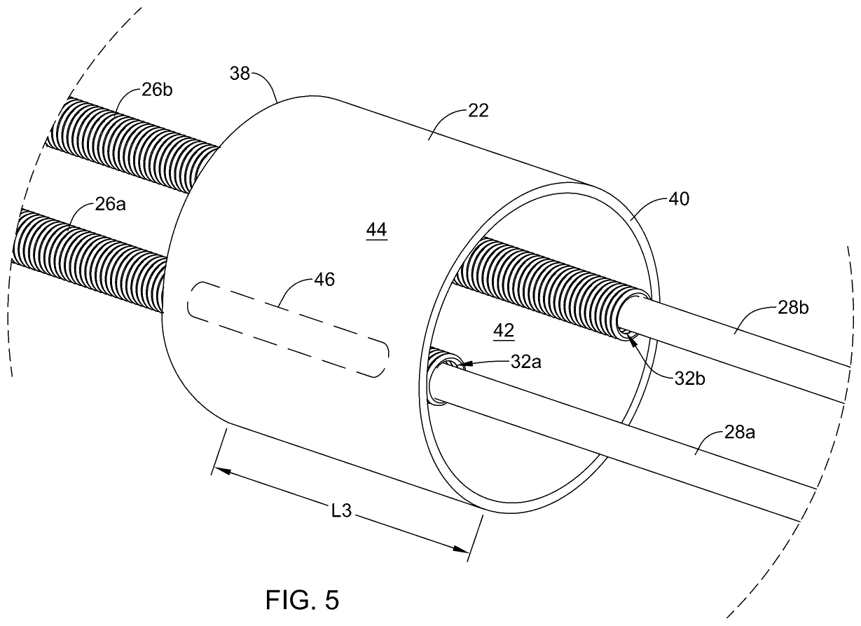

FIG. 5 is an enlarged view of a portion of FIG. 4, centered on the push ring 22. The push ring 22 may be considered as having a proximal edge 38 and a distal edge 40. The push ring 22 has an inner surface 42 and an outer surface 44. The outer surface 44 is adapted to contact a corresponding inner surface of the outer sheath 34. In some instances, depending on a length L₃ of the push ring 22, it may be possible to form one or more polymeric layers exterior to the push ring 22, including for example the outer sheath 34 (FIG. 3) without requiring the use of a mandrel.

The push ring 22 may be a metallic cylinder, for example. Suitable materials for the push ring 22 include stainless steel. In some instances, the push ring 22 may be a cylinder having a length L₃ in a range of 0.03 inches to 0.35 inches, an inner diameter in a range of 0.02 inches to 0.25 inches and an outer diameter in a range of 0.03 inches to 0.35 inches. The pull ring 24 may be a metallic cylinder, for example. Suitable materials for the pull ring 24 include stainless steel. In some instances, the pull ring 24 may be a cylinder having a length in a range of 0.03 inches to 0.35 inches, an inner diameter in a range of 0.02 inches to 0.25 inches and an outer diameter in a range of 0.03 inches to 0.35 inches.

In some instances, the push members 26a and 26b may be welded to the inner surface 42 of the push ring 22. A weld area 46 is shown in phantom, showing where the push member 26a is welded to the push ring 22. Any of a variety of welding techniques are contemplated. While not shown, a similar weld area on an opposing side of the push ring 22 is where the push member 26b is welded to the push ring 22.

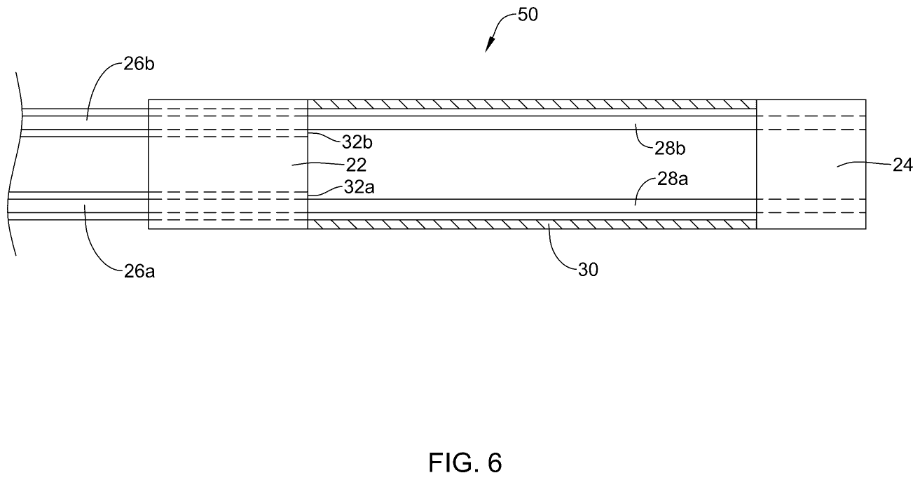

FIG. 6 is a schematic cross-sectional view of an illustrative steering assembly 50 that is adapted for use in a steerable catheter (such as the steerable catheter 10), the steerable catheter including an outer sheath (such as the outer sheath 34) defining an outer sheath lumen (such as the outer sheath lumen 36) extending therethrough, the steering assembly 50 being adapted to fit within the outer sheath lumen. The steering assembly includes the push ring 22 and the pair of push members 26a and 26b that are welded to the push ring 22 and that are adapted to extend proximally therefrom. Each of the push members 26a and 26b include the push member lumen 32a and 32b, respectively, extending therethrough. The steering assembly 50 includes the pull ring 24 and a pair of pull wires 28a and 28b that are welded to the pull ring 24 and that are adapted to extend proximally from the pull ring 24. A force transmission member 30 extends between the push ring 22 and the pull ring 24. The pull wire 28a extends through the push member lumen 32a within the push member 26a and the pull wire 28b extends through the push member lumen 32b within the push member 26b.

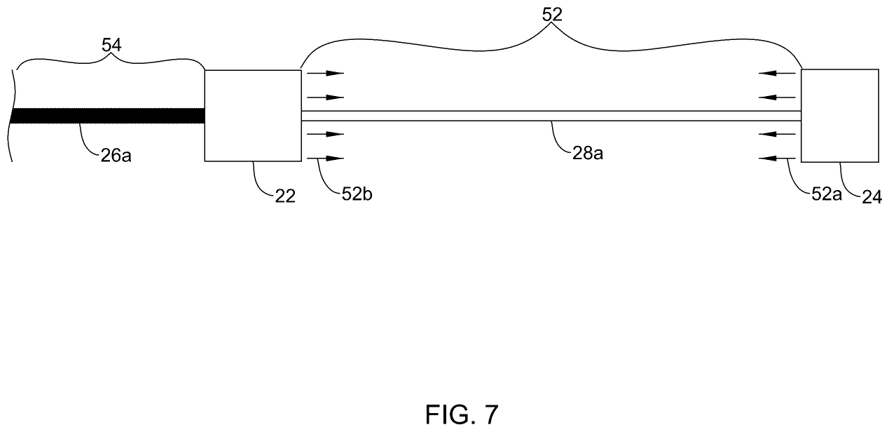

FIG. 7 is a schematic view showing how force transmission is influenced by the combination of the push ring 22 (and push member 26a) and the pull ring 24 (and pull wire 28a). As can be seen, the combination of the push ring 22 and the pull ring 24 results in a compression zone 52 that exists between the push ring 22 and the pull ring 24. The pull ring 24 is being urged proximally by pulling one or both of the pull wires 28a and 28b (only the pull wire 28a is visible in this view) proximally, as indicated by arrows 52a, and this compressive force is resisted by the push ring 22 not moving, held in place as indicated by arrows 52b, at least partially as a result of being embedded within the outer sheath 34 (FIG. 3) but also as a result of the push members 26a and 26b (only the push member 26a is visible in this view) being embedded in a polymeric layer and thus resistant to movement. A Bowden cable shielded zone 54, in which the push members 26a and 26b are embedded in polymer, serves to help anchor the push ring 22 and thus help form and maintain the compression zone 52.

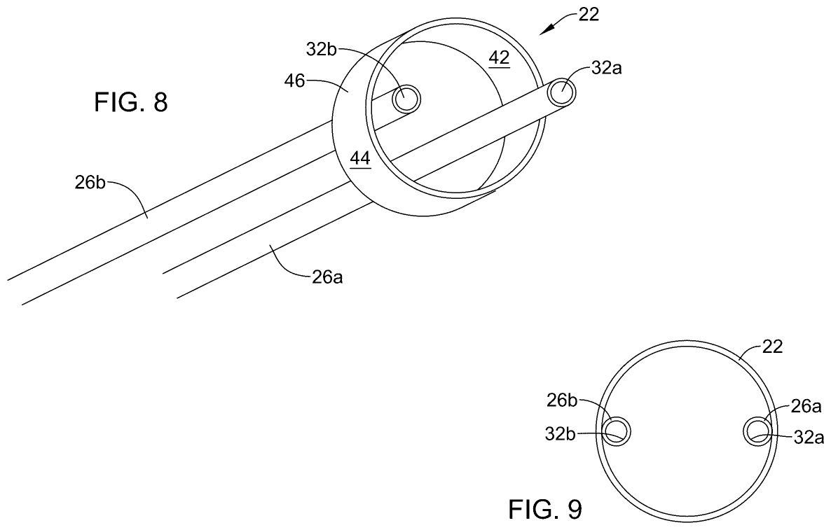

FIG. 8 is a perspective view, showing the push members 26a and 26b welded to the inner surface 42 of the push ring 22. This view clearly shows the push member lumens 32a and 32b extending through the push members 26a and 26b, respectively. In FIG. 8, the push members 26a and 26b are entirely within a circle defined by the inner surface 42 of the push ring 22. This is illustrated in FIG. 9, which is an end view. In some instances, the push members 26a and 26b may not be disposed entirely within the circle defined by the inner surface 42 of the push ring 22. In some instances, the push ring 22 may be notched or otherwise cut out to allow the push members 26a and 26b to be welded to the push ring 22, but in a position in which the push members 26a and 26b are moved radially outwardly in order to reduce the loss of volume inside the push ring 22 that would otherwise be consumed by the push members 26a and 26b.

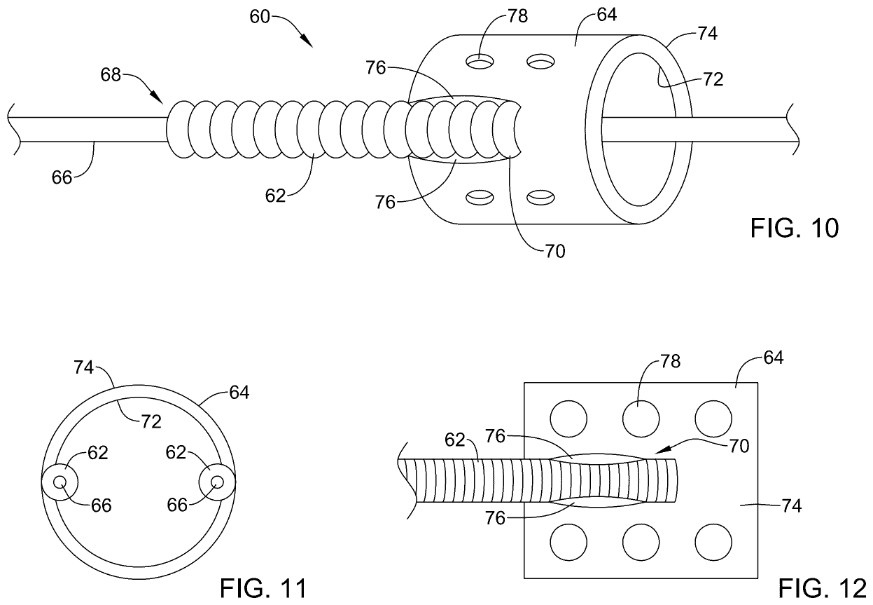

FIG. 10 is a schematic view of an illustrative assembly 60 in which a push member 62, representative of either the push member 26a or the push member 26b, is secured to a push ring 64 (representative of the push ring 22). FIG. 11 is an end view thereof, and FIG. 12 is a side view. A pull wire 66, representative of either the pull wire 28a or the pull wire 28b, extends through a push member lumen 68 extending through the push member 62. In some instances, the push ring 64 may include a notch 70 cut into the push ring 64 that enables the push member 62 to be moved radially outwardly from simply being positioned on the inner surface 42 (as seen in FIG. 5). Rather, the notch 70 allows the push member 62 to be positioned at least partially within the wall of the push ring 64. The push ring 64 may be considered as including an inner surface 72 and an outer surface 74. The push member 62 may extend radially inwardly beyond the inner surface 72, but not as far as it would without the notch 70. As seen in FIG. 11, the push member 62 extends radially inwardly beyond the inner surface 72, and extends radially outwardly to the outer surface 74. In some instances, the push member 62 may be positioned relative to the push ring 64 that the push member 62 extends radially outwardly beyond the outer surface 74.

The push member 62 is secured to the push ring 62 via one or more welds 76. In some instances, the push ring 62 may include one or more apertures 78 that extend from the inner surface 72 to the outer surface 74. In some instances, the one or more apertures 78 may be adapted to permit polymer reflow into the one or more apertures 78 when forming a steerable catheter, such as when forming an outer sheath that extends over the push ring 62. In some instances, polymer reflow into the one or more apertures 78 may help in anchoring the push ring 62. The push ring 62 may include any number of apertures 78.

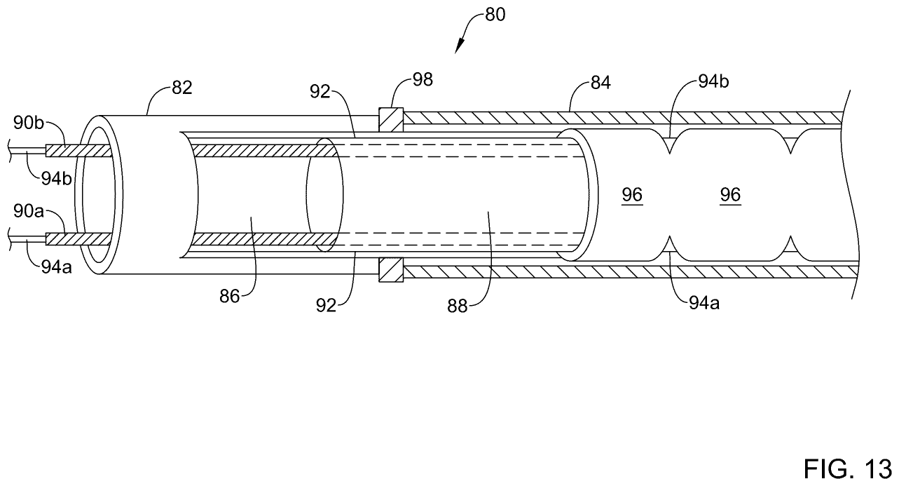

FIG. 13 is a schematic, partially cutaway, view of an illustrative steerable catheter 80. The illustrative steerable catheter 80 includes a proximal outer sheath 82 and a distal outer sheath 84. In some instances, the proximal outer sheath 82 and the distal outer sheath 84 may be formed of separate polymers. In some instances, the proximal outer sheath 82 and the distal outer sheath 84 may be formed of the same polymer. The proximal outer sheath 82 and the distal outer sheath 84 together define an outer sheath lumen 86 extending therethrough. A push ring 88 is disposed within the outer sheath lumen 86. A first push member 90a and a second push member 90b extend distally into the push ring 88 and are secured thereto via welds 92. A first pull wire 94a and a second pull wire 94b extend through the push members 90a and 90b, respectively, and extend distally therefrom.

The steerable catheter 80 includes a number of articulating joints 96 that are disposed within the outer sheath lumen 86. In some instances, the number of articulating joints 96 extend between the push ring 88 and a pull ring (not seen in this view) that is positioned distally of the number of articulating joints 96. In some instances, the number of articulating joints 96 may be considered as taking the place of the inner shaft member 30, and the number of articulating joints 96 may be considered together as forming a force transmission member extending between the push ring 88 and the unseen pull ring. Each of the articulating joints 96 may include internal structure that allows the pull wires 94a and 94b to extend through the articulating joints 96 while constraining the relative radial position of the pull wires 94a and 94b relative to the articulating joints 96. In some instances, the steerable catheter 80 may include a marker band 98. In some instances, the marker band 98 may denote where the proximal outer sheath 82 end and the distal outer sheath 84 begins, but this is not required.

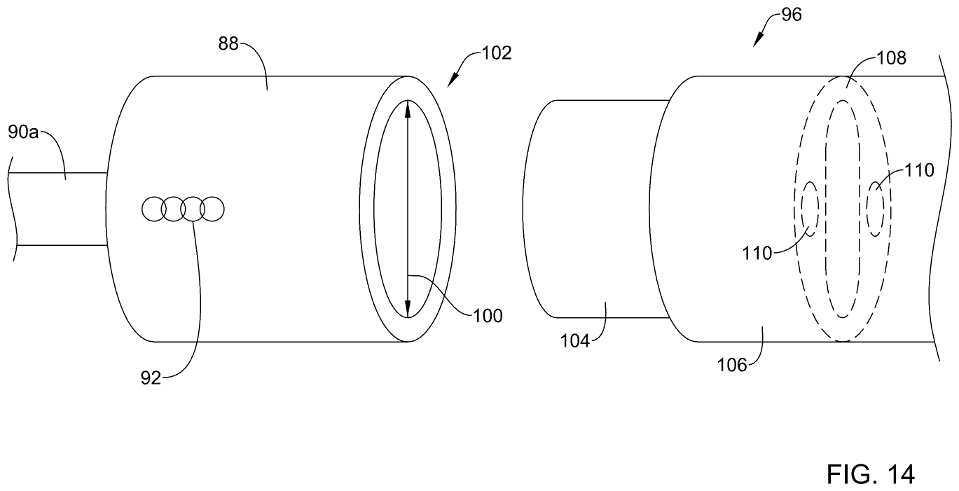

FIG. 14 provides a schematic view of a possible intersection between the push ring 88 and a first articulating joint 96. In some instances, the push ring 88 has an inner surface 100 defining an annular opening 102 into the push ring 88. The first articulating joint 96 may be considered as including a proximal segment 104 and a distal segment 106. The proximal segment 104 may have a diameter that is reduced relative to that of the distal segment 106. In some instances, the proximal segment 104 may have a diameter that allows the proximal segment 104 to fit into the annular opening 102 into the push ring 88. In some instances, the first articulating joint 96 (and each subsequent articulating joint 96) may include an internal structure 108 that helps to constrain and locate the pull wires (not shown) extending through the articulating joint 96. In some instances, the internal structure 108 may include a pair of apertures 110 through which the pull wires can extend.

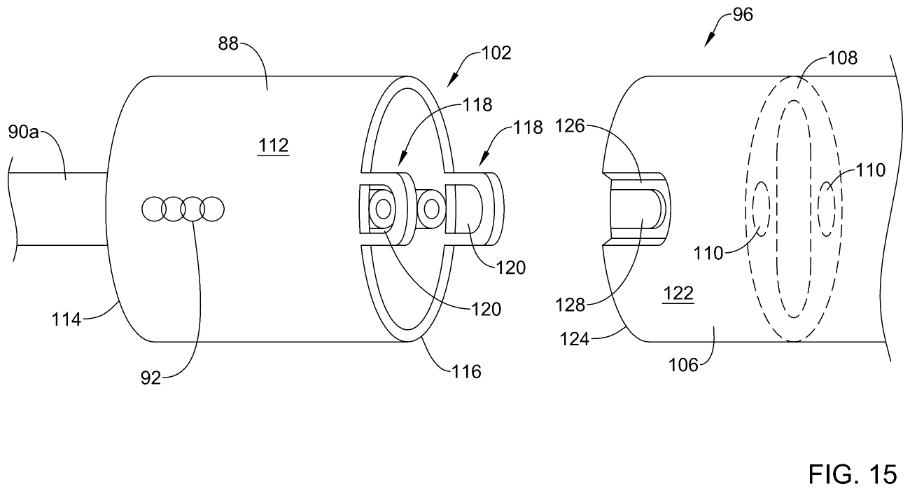

FIG. 15 provides a schematic view of a possible intersection between the push ring 88 and the first articulating joint 96. In some instances, the push ring 88 has an outer surface 112 that extends from a proximal edge 114 to a distal edge 116 of the push ring 88. In some instances, the push ring 88 may include securement features 118 that extend proximally from the distal edge 116. In some instances, the securement features 118 include an elongate aperture 120 that are adapted to engage complementary structure on the first articulating joint 96.

In some instances, the first articulating joint 96 includes an outer surface 122 extending distally from a proximal edge 124 of the first articulating joint 96. The outer surface 122 includes a recessed area 126. Angled tabs 128 extends radially outwardly from the recessed area 126 (only one is visible in this view), but in some instances does not extend radially outwardly from the outer surface 122 of the first articulating joint 96. It will be appreciated that when moving the push ring 88 towards the first articulating joint 96, the securement features 118 extending distally from the distal edge 116 of the push ring 88 will fit into the recessed area 126. The securement features 118 will extend over the angled tab 128 until the push ring 88 and the first articulating joint 96 are close enough together for the angled tabs 128 to snap into the elongate apertures 120, thereby securing the push ring 88 to the first articulating joint 96. The securement features 118 may be considered as being a distally extending first snap fit feature and the recessed areas 120 and angled tabs 128 may each together be considered as being a complementary second snap fit feature adapted to form a snap fit with the distally extending first snap fit feature.

The materials that can be used for the various components of the medical stent(s), the mandrel, and the various elements thereof disclosed herein may include those commonly associated with medical devices and mandrels. For simplicity purposes, the following discussion refers to the apparatus. However, this is not intended to limit the devices and methods described herein, as the discussion may be applied to other elements, members, components, or devices disclosed herein, such as, but not limited to, the medical stent, the mandrel, the filaments, the anti-migration loops, the covering, and/or elements or components thereof.

In some embodiments, the apparatus, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the apparatus, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some embodiments, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); antiproliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, antiplatelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative embodiments of the present disclosure, those of skill in the art will readily appreciate that yet other embodiments may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A steerable catheter, comprising:
an outer sheath defining an outer sheath haft lumen extending from a proximal region to a distal region;
a push ring secured within the proximal region of the outer sheath lumen;
a pull ring secured within the distal region of the outer sheath lumen;
an inner shaft member extending within the outer sheath lumen between the push ring and the pull ring;
a pair of push members extending through the outer sheath lumen and secured to the push ring, each of the pair of push members defining a push member lumen extending therethrough; and
a pair of pull wires secured to the pull ring, each of the pair of pull wires extending through one of the push member lumens.

2. The steerable catheter of claim 1, wherein each of the pull wires extend within the outer sheath lumen proximal of the pair of push members.

3. The steerable catheter of claim 1, wherein each of the pull wires extend within the outer sheath lumen distal of the pair of push members.

4. The steerable catheter of claim 1, wherein the push ring comprises a metal cylinder and each of the push members are welded to the metal cylinder.

5. The steerable catheter of claim 1, wherein each of the push members comprise a tightly wound coil.

6. The steerable catheter of claim 5, wherein each of the pull wires, extending through an associated push member, together with the associated push member, comprises a Bowden cable.

7. The steerable catheter of claim 1, wherein the push ring defines an inner diameter, and each push member of the pair of push members are disposed inside of the inner diameter.

8. The steerable catheter of claim 1, wherein the push ring defines an inner surface, and each push member of the pair of push members are disposed radially inwardly from the inner surface.

9. The steerable catheter of claim 1, wherein the push ring comprises a plurality of apertures for securing the push ring to the outer sheath.

10. The steerable catheter of claim 1, wherein the inner shaft member comprises a plurality of articulation joints.

11. The steerable catheter of claim 10, wherein a first articulation joint proximate the push ring includes a proximal reduced diameter segment adapted to fit inside of the push ring.

12. The steerable catheter of claim 10, wherein the push ring comprises a distally extending first snap fit feature and a first articulation joint proximate the push ring comprises a complementary second snap fit feature adapted to form a snap fit with the distally extending first snap fit feature.

13. A steering assembly adapted for use in a steerable catheter including an outer sheath defining an outer sheath lumen extending therethrough, the steering assembly adapted to fit within the outer sheath lumen, the steering assembly comprising:
a push ring;
a pair of push members welded to the push ring and adapted to extend proximally therefrom, each of the pair of push members defining a push member lumen extending therethrough;
a pull ring;
a pair of pull wires welded to the pull ring and adapted to extend proximally from the pull ring; and
a force transmission member extending between the push ring and the pull ring;
wherein the pair of pull wires each extend through a corresponding push member lumen proximal of the push ring.

14. The steering assembly of claim 13, wherein each of the push members comprise a tightly wound coil.

15. The steering assembly of claim 14, wherein each of the pull wires, extending through an associated push member, together with the associated push member, comprises a Bowden cable.

16. The steering assembly of claim 13, wherein the push ring comprises a plurality of apertures for securing the push ring to the outer shaft.

17. The steering assembly of claim 13, wherein the force transmission member comprises a plurality of articulation joints.

18. The steering assembly of claim 17, wherein a first articulation joint proximate the push ring includes a proximal reduced diameter segment adapted to fit inside of the push ring.

19. The steering assembly of claim 17 wherein the push ring comprises a distally extending first snap fit feature and a first articulation joint proximate the push ring comprises a complementary second snap fit feature adapted to form a snap fit with the distally extending first snap fit feature.

20. A steerable catheter, comprising:
an outer shaft defining an outer shaft lumen extending from a proximal region to a distal region;
a push ring secured within the proximal region of the outer shaft lumen;
a pull ring secured within the distal region of the outer shaft lumen;
a plurality of articulation joints extending within the outer shaft lumen between the push ring and the pull ring;
a pair of push members extending through the outer shaft lumen and welded to the push ring, each of the pair of push members defining a push member lumen extending therethrough; and
a pair of pull wires welded to the pull ring, each of the pair of pull wires extending through one of the push member lumens, each of the pair of pull wires extending through the outer shaft lumen both proximal and distal of the pair of push members.
